(19) United States
(12) Patent Application Publication
(10) Pub. No.: US 2018/0345040 A1
(43) Pub. Date: Dec. 6, 2018

(54) A TARGET SURFACE

(71) Applicant: VISION RT LIMITED, London (GB)

(72) Inventors: Ivan MEIR, London (GB); Edward MEAD, London (GB)

(21) Appl. No.: 15/573,825

(22) PCT Filed: May 12, 2016
(86) PCT No.: PCT/GB2016/051372

§ 371 Date: Nov. 13, 2017

(30) Foreign Application Priority Data
May 13, 2015 (GB) 1508163.1

(51) Int. Cl. A61N 5/10; G06T 7/80; G06T 7/00; H04N 13/282; H04N 13/246

(52) U.S. Cl. CPC A61N 5/1049; G06T 7/85; G06T 7/0012; A61B 2090/3937; H04N 13/246; A61N 2005/1059; A61N 2005/1061; H04N 13/282

(57) ABSTRACT

Some embodiments are directed to a target surface including one or more three-dimensional projections, each projection including a multiplicity of planar side surfaces, a stereoscopic camera operable to obtain images of the target surface, and a processing module operable to process images obtained by the stereoscopic camera of the target surface together with data identifying the position and orientation of the stereoscopic camera relative to a defined point in space to determine the positions relative to the defined point in space of the planar side surfaces defined by the at least one or more three-dimensional projections.

### CROSS REFERENCE TO RELATED APPLICATION

This application is a National Phase filing under 35 C.F.R. § 371 of and claims priority to PCT Patent Application No.: PCT/GB2016/051372, filed on May 12, 2016, which claims the priority benefit under 35 U.S.C. § 119 of British Application No.: 1508163.1, filed on May 13, 2015, the contents of which are hereby incorporated in their entireties by reference.

### BACKGROUND

The presently disclosed subject matter relates to a target surface. Some embodiments therefore relate to a target surface for using in patient positioning and monitoring during radiotherapy.

Radiotherapy (RT) can include or can consist of projecting, onto a predetermined region of a patient's body, a radiation beam so as to destroy or eliminate tumors existing therein. Such treatment is usually carried out periodically and repeatedly. At each medical intervention, the radiation source must or should be positioned with respect to the patient in order to irradiate the selected region with the highest possible accuracy.

Known RT apparatuses are calibrated such that a generated radiation beam is focused on what is referred to as the treatment iso-center and patient monitoring systems are employed to monitor a patient's position to ensure the iso-center coincides with a tumor being treated. Such monitoring systems often include one or more stereoscopic cameras able to track a patient's position. If a patient lies on a mechanical couch then under the control of the monitoring system the mechanical couch positions the patient in the correct position such that the iso-center is focused on the tumor.

The stereoscopic cameras monitor natural features on the patient's body or physical markers applied to the surface of the patient. The cameras are generally in fixed locations suspended from the ceiling of a treatment room located 1.5 to 2 m away from the patient. Being in a fixed position enables the cameras to be calibrated so as to identify the relative position of a patient relative to the treatment iso-center. At the same time, being remote from the patient, the cameras do not get in the way of the treatment apparatus itself.

For stereotactic surgery, in particular when treating brain tumors, it is essential that the patient is positioned relative to the RT delivery system with very high accuracy so that radiation is delivered to the tumor, and not the surrounding healthy tissue. For this reason, the head of a patient undergoing stereotactic surgery is securely attached to a couch via a frame or a face mask so that the patient cannot move their head during treatment.

### SUMMARY

Although related patient monitoring systems are able to monitor patients with high accuracy, further improvements with which such systems can monitor patients may be desirable.

Some embodiments provide a a target surface having one or more three-dimensional projections provided thereon, each projection having a multiplicity of planar side surfaces.

Imaging a three-dimensional projection having a multiplicity of planar side surfaces utilizing a stereoscopic camera enables a model of the surface of the three-dimensional projection to be created. Where it is known that a three-dimensional projection has a multiplicity of planar side surfaces, planes of best fit for the planar surfaces can be determined and hence the position and orientation of the surfaces.

In one embodiment, the planar side surfaces on each of the three-dimensional projections converge to define a point and the processing module is operable to determine the positions relative to the defined point in space of the point features defined by the at least one or more three-dimensional projections.

The point of intersection of planes of best fit corresponding to the planar surfaces thus enables the position of an identified point to be determined with high accuracy.

The use of three-dimensional projections having a multiplicity of planar side surfaces converging to define a point feature means that the identification of the location of a point feature can be determined from measurements of a plurality of points on the planar surfaces. Thus the measurement of the position of the point feature is dependent upon a large number of data measurements and hence less liable to error.

Further, the determination of planes corresponding to the planar surfaces can provide data indicative of the relative orientation of the target surface.

In one embodiment the target surface is provided on a head mounting frame enabling the position and orientation of a patient's head to be determined.

Where a plurality of three-dimensional projections are provided, the relative positions of the point features identified by the plurality of three-dimensional projections enables the orientation of the target surface to be determined.

Projections of different heights are provided or projections are arranged in an asymmetric pattern. This is possible because it simplifies the identification of different projections and means that the orientation of a target surface can be uniquely determined.

In some embodiments two target surfaces may be provided circumferentially spaced apart. Providing at two or more target surfaces increases the likelihood that at least one of the target surfaces will be visible to a stereoscopic camera.

### BRIEF DESCRIPTION OF THE DRAWINGS

Some embodiments will now be described by way of example only with reference to the accompanying drawings, in which:

FIG. 1 is a perspective view of a treatment system including a monitoring system according to an embodiment of the presently disclosed subject matter,

FIG. 2 is a schematic side view of the system of FIG. 1,

FIG. 3 is a perspective view of a head frame to which exemplary target surfaces to be monitored are attached, and

FIG. 4 is a perspective view of an alternative target surface.

### DETAILED DESCRIPTION OF THE PREFERRED EMBODIMENTS

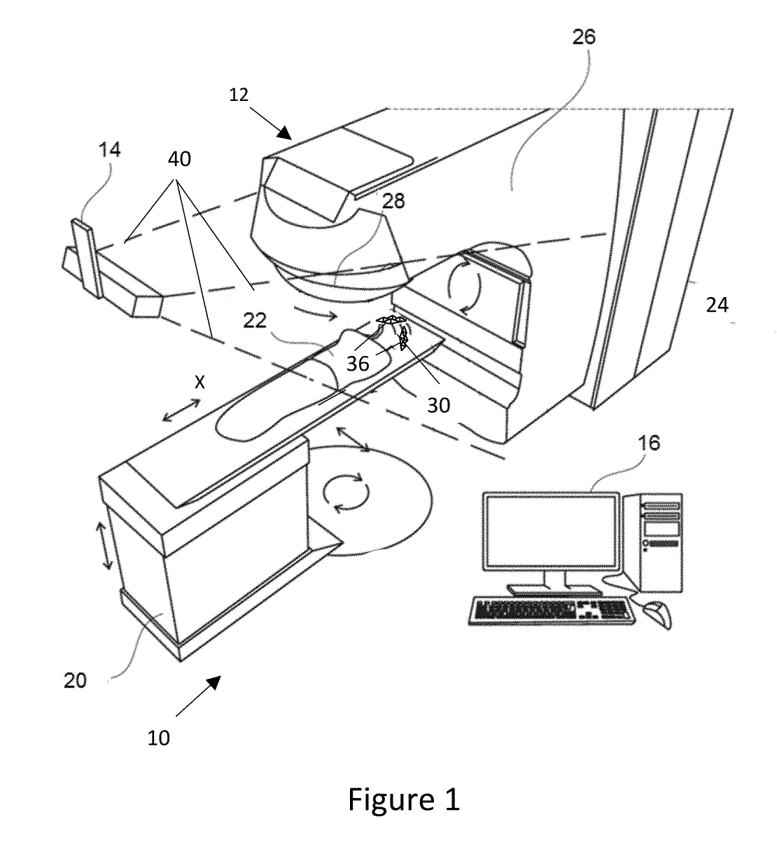

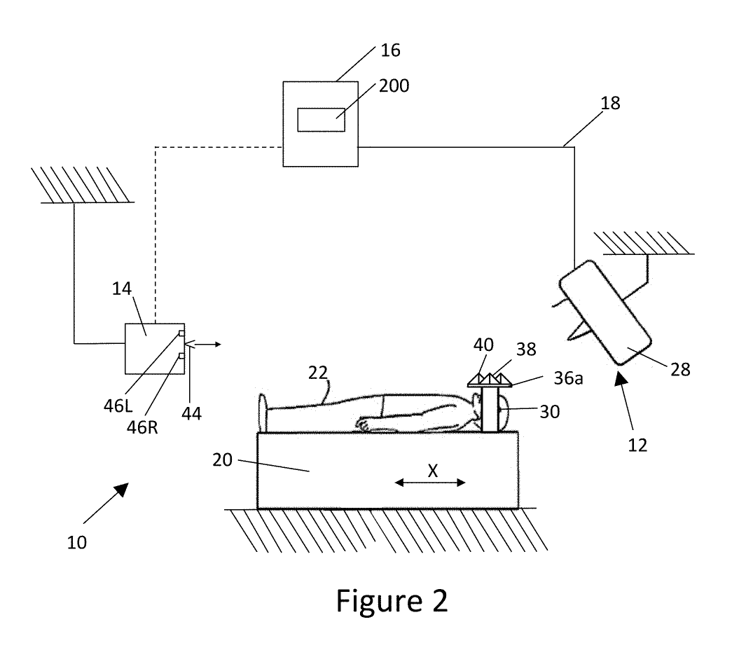

In FIGS. 1 and 2, a treatment system 10 includes a treatment apparatus 12 such as a linear accelerator for applying radiotherapy or an x-ray simulator for planning radiotherapy, a stereoscopic camera 14, and a computer 16. In this embodiment the stereoscopic monitoring camera 14 is suspended from the ceiling of the treatment room, a distance away (e.g., 1.5-2 m) from the patient and the treatment apparatus 12.

The camera 14 is connected to the computer 16 wirelessly (shown by a dashed line in FIG. 2). The camera can also be connected to the computer 16 via a physical wire. The computer 16 is also connected to the treatment apparatus 12 via a wire 18.

A mechanical couch 20 is provided upon which a patient 22 lies during treatment. The treatment apparatus 12 and the mechanical couch 20 are arranged such that under the control of the computer 16, the relative positions of the mechanical couch 20 and the treatment apparatus 12 may be varied, laterally, vertically, longitudinally and rotationally.

As may be seen in FIG. 1, the treatment apparatus 12 includes a main body 24 from which extends a gantry 26. A collimator 28 is provided at the end of the gantry 26 remote from the main body 24 of the treatment apparatus 12. To vary the angles at which radiation irradiates a patient 22, the gantry 26, under the control of the computer 16, is arranged to rotate about an axis passing through the center of the main body of the treatment apparatus 12. Additionally the location of irradiation by the treatment apparatus may also be varied by rotating the collimator 28 at the end of the gantry 26.

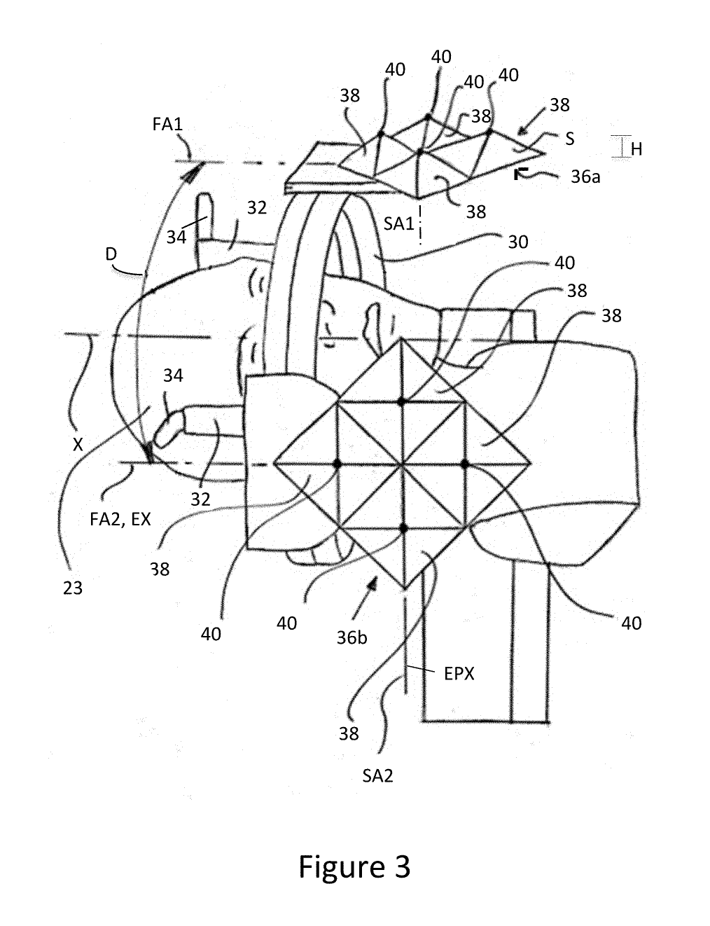

FIG. 3 is a perspective view of a head frame 30 to which exemplary target surfaces to be monitored are attached, FIG. 3 shows a ring-shaped head mounting frame 30 which is secured to a patient 22 and then is secured to the mechanical couch 20 causing the patient to hold their head in a fixed position and orientation relative to the mechanical couch. The head mounting frame 30 has a longitudinal axis X and includes projections 32 extending from the mounting frame 30 generally in the direction of the longitudinal axis X, and securing screws 34 which secure a head 23 of the patient 22 undergoing treatment.

A first target surface 36a and a second target surface 36b are secured to the head mounting frame 30. The first 36a and second 36b target surfaces are circumferentially space by a distance D around the longitudinal axis (X). The first target surface 36a has a first axis FA1 and a second axis SA1 which is perpendicular to the first axis FA1. The second target surface 36b has a first axis FA2 and a second axis SA2 which is at angle θ, in this embodiment, perpendicular to the first axis FA2. The first axis FA1 of the first target surface 36a is parallel to the first axis FA2 of the second target surface 36b. In alternative embodiments the target surfaces can be arranged differently, the key requirement being that taking into account the possible positions of the mechanical couch and gantry, enough of the target surfaces can be imaged by the stereoscopic camera to enable the surfaces to be accurately tracked.

Each target surface 36a,36b in this embodiment has four identical square based pyramids 38 provided thereon. Each pyramid 38 has a square base and side surfaces S of equal area. The pyramids 38 are arranged symmetrically in a square matrix such that a line of edges EX extend in the direction of and parallel to the axis X of the target surface 36a,b and a line of edges EPX extend in a direction perpendicular to and spaced from the axis X. Each of the side surfaces S converge towards a point feature (the apex of the pyramid) 40 and have a height H.

As will be explained the target surfaces 36a,36b are such that the points corresponding to the apices 40 of the pyramids 38 can be identified with very high accuracy by the monitoring system, and thus the position and orientation of a patient's head can be monitored with very high accuracy during treatment.

Returning to FIGS. 1 and 2, the camera 14 is mounted on the ceiling at a distal position and has a field of view 40 (FIG. 1) which it can be seen ensures the camera 14 is in direct line-of-sight with the target surfaces 36a,b.

The camera 14 is a stereoscopic camera which is well known for use in monitoring objects such as treatment apparatus and patients in RT systems, and is therefore not described in detail herein save to say that a speckle projector 44 (FIG. 2) is integrated with the camera 14 (so as to form a module), and it generally includes two lenses 46L,46R which are positioned in front of image detectors such as CMOS active pixel sensors or charge coupled devices (not shown) contained within the module. The image detectors are arranged behind the lenses 46L,46R so as to capture images of the target surfaces 36a,b. The speckle projector 44 is positioned between the two lenses 46L,46R and is arranged to illuminate the pyramids 38 with a pseudo random speckle pattern of infrared light so that when images of pyramids 38 are captured by the two image detectors, corresponding portions of captured images can be distinguished. To that end, the speckle projector 44 includes a light source such as an LED and a film with a pseudo random speckle pattern printed on the film. In use, light from the light source is projected via the film and as a result a pattern including or consisting of light and dark areas is projected onto the surfaces S of pyramids 38. The captured images can then be processed to determine the position and orientation of a set of points on the surfaces of the pyramids 38.

In order for the computer 16 to process the images received from the camera 14, the computer 14 is configured by software either provided on a disk or by receiving an electrical signal via a communications network to a processing module 200. The processing module 200 is able to process the images from the camera 14 to determine the position and orientation of the pyramids 38 and therefore the patient 22.

The position and orientation of the head frame and hence the position and orientation of the patient 22 is obtained as follows:

Firstly the camera 14 is calibrated so as to be able to process images from the camera and to determine the position and orientation of objects captured in those images. In order to do so it may be necessary to determine various internal parameters for the cameras (e.g., focal length, any lens distortions etc.) so that images can be related to distances in the real world.

Briefly, a calibration object in the form of a calibration sheet such as a 40×40 cm sheet of flat rigid material such as aluminium or steel on which a pattern revealing a 20×20 matrix of circles at known positions on the surface of the sheet is provided. Additionally, towards the center of the calibration sheet are four smaller markers adjacent to four circles the centers of which together identify the four corners of a square of known size. Images of the calibration sheet are obtained and processed by a position determination module to identify within the image the positions of the four markers in the images and their associated circles. From the relative positions of circles identified by the markers in the images, a projective transformation is determined which accounts for the estimated centers of the identified circles defining the corners of a parallelogram in the image which arises due to the relative orientation of the calibration sheet and the lenses 46L,46R of the camera 14 obtaining the image. Optionally, and to further increase accuracy of the estimated positions of the centers of the circles, the calculated transform is then applied to each of the identified circles in turn to transform the oval shapes of the circles. The positions of the centers of the four circles are then determined by identifying the centers of the transformed circles and utilising an inverse transform to determine the corresponding position of the estimated circle center in the original image. When the coordinates for all or most of the centers of each of the representations of the circles on the calibration sheet have been calculated for an image, the relative orientation of the lenses of the tracking camera can then be calculated from the relative positions of these points in the images and the known relative locations of these circles on the surface of the calibration sheet as is described in detail in “A Versatile Camera Calibration Technique for High-Accuracy 3D Machine Vision Metrology Using Off the Shelf TV Cameras and Lenses”, Roger Tsai, IEEE Journal of Robotics and Automation, Vol. Ra-3, No. 4, August 1987. Further from the relative positions of the points in the individual images internal camera parameters such as the focal length and radial distortion within the camera images can also be determined.

Having determined the relative locations of the lenses of the camera 14 and any lens distortions present in the camera images, the next step is to determine the position and orientation of the camera 14 relative to the iso-center of the treatment apparatus 12.

This is achieved by imaging a calibration cube of known size which is positioned on the mechanical couch 20 of the treatment apparatus 12 at a position with its center at the iso-center of the treatment apparatus 12 as indicated by the co-incidence of marks on the exterior of the cube with the projection of the laser cross hairs which intersect at the iso-center. The images of the calibration cube are processed utilising the previously obtained measurements of the relative locations of the camera lenses and any data about the existence of any distortion present in the images to generate a 3D computer model of the surface of the cube. Since the cube has known dimensions and is at a known location and in a known orientation relative to the iso-center of the treatment apparatus as indicated by the laser cross-hairs, a comparison between the generated 3D model of the calibration cube, and the known parameters for the size and position of the calibration cube enables the position and orientation of the camera 14 to be determined relative to the iso-center such that subsequent position and orientation information determined relative to the camera 14 can be converted into position and orientation information relative to the treatment apparatus iso-center.

In the alternative, other approaches for determining the relative position and orientation of the camera 14 relative to the iso-center, for example, as described in WO2015/008040, the contents of which is herein incorporated by reference, may also be used.

Having determined the relative position and orientation of the camera 14 relative to the iso-center of the treatment apparatus 12, the camera 14 obtains images of the pyramids 38 of the target surfaces 36a,b. A suitable approach for converting images of a surface into a 3D model of the surface is described in Vision RT's U.S. Pat. No.7,889,906, the contents of which is herein incorporated by reference.

Briefly, images of the speckled pattern projected from the projector 44 of the camera 14 onto the surfaces S of the pyramids 38 on the head mounting frame 30 are obtained by the left 46L and right 46R lenses of the camera 14.

The processing module 200 then proceeds to determine transformations to identify and match corresponding portions of the images (typically the analysis is of image patches of around 16×16 pixels) received by the left 46L and right 46R lenses. Matching corresponding portions of these images together with knowledge of the relative locations of the image planes for the image detectors behind the left 46L and right 46R lenses enables locations corresponding to points on the pyramid surfaces S and the location of the point features 40 to be identified on each pyramid 38.

The locations of the points corresponding to the apices of the pyramids can then be determined. More specifically, the set of points corresponding to individual surfaces S of each pyramid can be determined. Allowing for errors, all or most of the points on a particular surface should lie on a common plane. The mathematical plane corresponding to the plane of best fit can be determined. Similar planes of best fit can be determined for other surfaces S and the point of intersection of those planes will uniquely identify the position of the apex of the pyramid.

It may be beneficial in providing a projection such as a pyramid 38 having a number of surfaces S meeting at a point 40, the location of that point 40 can be determined with very high accuracy because the position of the point 40 is inferred from multiple measurements of the speckled pattern projected onto the surfaces S of the pyramid 38.

It may also be beneficial in that providing a plurality of pyramids 38 further increases the accuracy as it increases the number of point features 40 which can be captured by the images detectors and processed. The same principle applies to providing projections on two target surfaces 36a,b. Furthermore, spacing the target surfaces 36a,b ensures that a sufficient number of point features 40 are visible in the event that the line-of-sight between the camera 14 and the target surfaces 36a,b is partially blocked.

Once known, the positions of the point features can be compared with stored reference data of the expected positions of the point features 40 if a patient is correctly positioned relative to the treatment iso-center. If there is alignment then the RT delivery can continue. If there is not alignment then the computer 16 can output patient movement instructions to move the mechanical couch 20 to position the patient 22 relative to the RT delivery to make sure the iso-center is located on the tumor, or to halt treatment.

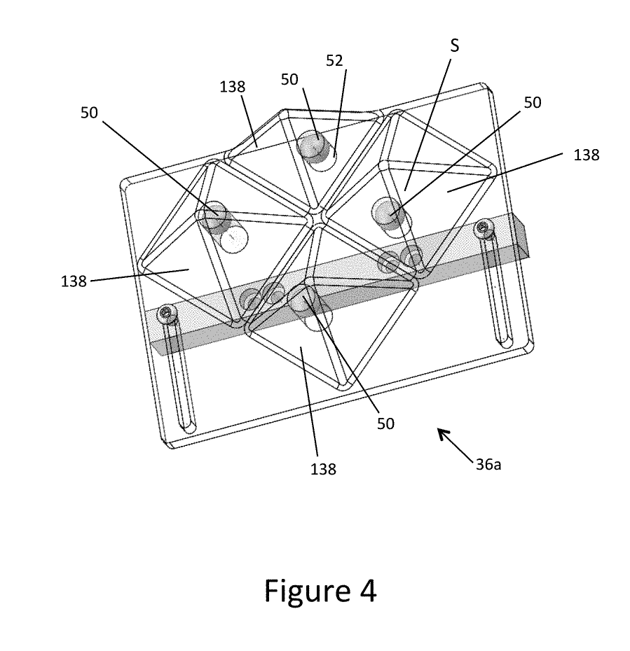

In FIG. 4, four alternative projections 138 are provided on each target surface 36a,36b. The projections 138 are identical to those described in relation to FIGS. 1 to 3 except that a sphere 50 of a radiopaque material such as tungsten is embedded within a channel 52 of each projection 138. The spheres 50 are arranged such that they can be individually distinguished from any angle when being imaged, specifically such that the spheres may never be superimposed on one another during a computed tomography (CT) scan, the purpose of which will be described below.

Prior to the patient undergoing radiation treatment, reference volumetric images of the patient are obtained by performing a CT scan. These images are used to accurately determine the position of tumors within the patient and enable planning of the radiation treatment to ensure the radiation beam is focused on the tumors and not surrounding tissue. Given that the pyramids 138 and the spheres 50 are manufactured to tight manufacturing tolerances, and that precise measurements are obtainable by using a coordinate measure machine, the position of the spheres 50 relative to the surface S of the pyramids 138 can be determined with a high degree of accuracy. The images of the spheres 50 are more distinguishable than if images of the outline of the pyramids 138 were obtained during the CT scan, and therefore the position of the tumors within the patient relative to the spheres 50, and hence relative to the surface S due to their known fixed relationship can be obtained with a high degree of accuracy.

When the patient undergoes radiotherapy, since the position of the surface S of the pyramid 138 relative to the iso-center is known as well as the position of the camera 14 relative to the iso-center (through calibration as described above), the position of the tumors relative to the iso-center is also known having determined the positional relationship between the surface S of the pyramids 138 and the spheres 50 during the CT scan. Knowing the position of the tumor relative to the iso-center of the treatment apparatus enables radiation to be applied to the tumor.

In some embodiments, the pyramids 38, and hence the point features 40 are arranged in a symmetric pattern. In an alternative embodiment the pyramids may be arranged in an asymmetric pattern or have different heights. Providing an asymmetric pattern or pyramids may be advantageous as it facilitates the identification of individual pyramids when processing image data.

Although in the above described embodiment, square based pyramids are described, the presently disclosed subject matter need not be limited to square based pyramids, for example, a triangle based pyramid could be provided on the target surface.

Some embodiments describe determining the position of the apices of the pyramids from multiple measurements of the speckled pattern projected onto the surfaces S of the pyramids. In an alternative embodiment, multiple measurements of the speckled pattern on two surfaces of the pyramids are processed to determine the position of those surfaces rather than the apices of those intersecting surfaces.

In an alternative embodiment, the pyramids need not have apices defined by a point, for example they can have rounded apices. Similarly, the planar surfaces can have rounded edges where they meet. It will be appreciated that the mathematical modelling of such rounded edges/apices enables their position to be determined despite the absence of a physical point feature or edge.

The target surfaces described in the above embodiments are provided on a head mounting frame 30 which enables the position and orientation of the head 23 of the patient 22 to be determined which is essential in stereotactic surgery. It will be appreciated that the target surface need not be limited to being provided on the head mounting frame 30, and can be provided on any object whose position and orientation needs to be monitored with high accuracy.

1. A target comprising:
one or more three-dimensional projections, each projection including a multiplicity of planar side surfaces.

2. (canceled)

3. The target surface according to claim 1, wherein the planar side surfaces on each of the three-dimensional projections converge to define a point feature, wherein a processing module is operable to determine the positions relative to the defined point in space of the point features defined by the at least one or more three-dimensional projections.

4. The target surface according to claim 1 further including an object to be monitored, and including a longitudinal axis, the surface being secured to the object, and movable therewith.

5. The target surface according to claim 1, wherein the at least one three-dimensional projection is a plurality of three-dimensional projections.

6. The target surface according to claim 5, w herein one of the plurality of three-dimensional projections has a volume which differs from another of the plurality of three-dimensional projections.

7. The target surface according to claim 5, wherein the one of the plurality of three-dimensional projections has a height which differs from another of the plurality of three-dimensional projections.

8. The target surface according to claim 5, wherein the three-dimensional projections are arranged in an asymmetric pattern.

9. The target surface according to claim 1, wherein the target surface include a first target surface and a second target surface.

10. The target surface according to claim 8, wherein the first and second target surfaces are circumferentially spaced on the object around the longitudinal axis.

11. The target surface according to claim 8, wherein the first and second target surfaces each have first axes extending parallel to each other and second axes extending at an angle relative to their respective first axes.

12. The target surface according to claim 10, wherein the first axes extend perpendicular to their respective second axes.

13. The target surface according to claim 10, wherein first axes each extend in a direction parallel to the longitudinal axis of the object.

14. The target surface according to claim 1, further including a projector for projecting a pre-defined pattern onto the at least one target surface.

15. The target surface according to claim 1, wherein the at least one three-dimensional projection is a pyramid.

16. The target surface according to claim 14, wherein the pyramid is a square based pyramid.

17. A radiotherapy treatment system, comprising:
a treatment apparatus; and
the target surface according to claim 20.

18. (canceled)

19. (canceled)

20. A monitoring system for use with a radiotherapy apparatus, comprising:
the target surface according to claim 1; and
a 3D camera operable to obtain images of the target surface together with data identifying the position and orientation of the 3D camera relative in a defined point in space to determine the positions relative to the defined point in space of the planar side services defined by the at least one or more three-dimensional projections.
